(19) United States
(12) Patent Application Publication
(10) Pub. No.: US 2021/0023279 A1
(43) Pub. Date: Jan. 28, 2021

(54) SYSTEMS INCLUDING EXTERNAL CATHETER FOR AUTOMATICALLY COLLECTING URINE FROM A FEMALE PATIENT AND METHODS OF USE

(71) Applicant: Boehringer Technologies, LP, Phoenixville, PA (US)

(72) Inventors: Christopher L. Radl, Malvern, PA (US); Michael Reed Vennel, Phoenixville, PA (US)

(21) Appl. No.: 16/921,084

(22) Filed: Jul. 6, 2020

Related U.S. Application Data
(60) Provisional application No. 62/877,527, filed on Jul. 23, 2019.
(60) Provisional application No. 62/882,761, filed on Aug. 5, 2019.

(51) Int. Cl. A61M 1/00; A61F 5/455; A61M 39/10; A61M 39/22

(52) U.S. Cl. CPC A61M 1/0035; A61F 5/455; A61M 1/0033; A61M 2039/226; A61M 39/22; A61M 2202/0496; A61M 2039/1077; A61M 39/10

(57) ABSTRACT

System and methods for automatically removing by suction urine voided by a patient, e.g., a female. The systems include an external catheter, a suction canister and a sound suppressed suction regulator. The suction regulator and canister may form an integral unit. The external catheter is applied at the urethra opening to receive urine voided by the patient. The suction canister collects the urine and is coupled to a source of suction providing suction having a first value. The suction regulator is interposed between the external catheter and the canister to regulate the amount of suction to a regulated value which is applied the external catheter to carry urine from the external catheter into the canister. The suction regulator includes passageways through it that do not form a tortuous path so that noise is not generated by the air flow through those passageways.

### CROSS-REFERENCE TO RELATED APPLICATIONS

This utility application claims the benefit under 35 U.S.C. § 119(e) of Provisional Application Ser. No. 62/877,527, filed on Jul. 23, 2019, entitled “System Including External Catheter And Integrated Suction Regulator and Canister For Automatically Collecting Urine From A Patient And Method Of Use”, and Provisional Application Ser. No. 62/882,761, filed on Aug. 5, 2019, entitled System Including A Low Noise Suction Regulator For Automatically Removing Urine From A Patient Via An External Catheter”. The entire disclosures of those two provisional applications are incorporated by reference herein.

### FIELD OF THE INVENTION

This invention relates generally to medical devices and more particularly to devices for automatically removing urine from a patient, e.g., a female, using suction applied to an external catheter.

### BACKGROUND OF THE INVENTION

Various external catheters are available for non-invasive urine output management in female patients. The PUREWICK® female external catheter available from C.R. Bard, Inc. is an example of one such device. That external catheter is a soft member having a hollow flexible body including a side opening exposing soft absorbent gauze. The catheter is configured to be positioned so that soft gauze is disposed between the patient's separated gluteus and labia and in fluid communication with the urethral opening of the patient, whereupon urine voided by the patient is wicked into the gauze. The catheter is arranged to be attached via suction tubing to a suction canister, which should in turn be connected to either a suction regulator on a hospital wall or a portable suction pump, such as the DRYDOC™ vacuum suction station of C.R. Bard, Inc., whereupon the urine wicked into the external catheter is carried by the suction into the canister for collection. The Instructions for Use (IFU) of the PUREWICK® female external catheter indicates that the suction source should be set to a minimum of 40 mmHG continuous suction.

Sage Products, LLC, now a Stryker Corporation company, provides an external urine management system for females under the trademark PRIMAFIT. That system is in many respects similar to the PUREWICK® system. In particular, the PRIMAFIT system basically comprises an external catheter body having an end cap to fit in the woman's perineal area to secure the catheter in place. The catheter includes soft wicking fabric that absorbs and diverts urine away from the patient's skin. Urine is then absorbed into the system's core and suctioned into a collection canister.

The patented literature includes various systems and methods for collecting and transporting urine away from a person's body, such as: U.S. Pat. No. 4,610,675 (Triunfol); U.S. Pat. No. 4,747,166 (Kuntz); U.S. Pat. No. 5,678,564 (Lawrence et al.); U.S. Pat. No. 5,894,608 (Birbara); U.S. Pat. No. 6,849,065 (Schmidt et al.); U.S. Pat. No. 7,018,366 (Easter); U.S. Pat. No. 7,220,250 (Suzuki et al.); and U.S. Pat. No. 8,287,508 (Sanchez).

As will be appreciated by those skilled in the art, most hospital suction regulators provide insufficient flow at low vacuum pressures, like the 40 mmHg recommended for use with the PUREWICK® female external catheter. Therefore nurses or other care givers frequently increase the vacuum to get adequate urine flow. However, the use of higher vacuum pressure poses an increased risk to the patient, as the only opening in the circuit for air to relieve the pressure is adjacent the patient's genitalia. Accordingly, use of increased vacuum pressure to increase the flow rate of urine being withdrawn into the canister runs the risk of injury to the delicate issue adjacent the urethral opening.

Thus, a need exists for a system and method which is efficient for removing urine from a patient using an external catheter, wherein the flow rate is sufficiently high for increased effectiveness, yet is produced by a suction level that is sufficiently low to minimize the danger of injury to the delicate tissue of the patient adjacent the patient's urethral opening.

In our copending U.S. Non-Provisional Application Ser. No. 16/833,980, filed on Mar. 30, 2020, entitled “System Including Suction Regulator For Automatically Removing Urine From A Female Patient” (hereinafter the “'980 Application”), whose disclosure is incorporated by reference herein, there is disclosed and claimed a suction regulator and system making use of that suction regulator that addresses the above need. That system includes disposable suction regulator configured for use between the female external catheter and a canister coupled to a source of higher suction, e.g., a regulator at the hospital's suction line. The disposable female external catheter suction regulator of that provisional application is designed in such a way that it allows far greater flow at low pressures than do the traditional wall regulators. As such, it is intended to be placed in the same circuit, but between the external catheter and the canister. The exemplary embodiment of the suction regulator includes a tortuous passageway, e.g., a right angle or L-shaped passageway, coupled to the receptacle or canister and the source of suction. The flow of high velocity air through that tortuous path during operation of the suction regulator has been determined to produce unnecessary noise, e.g., a whistling sound. The '980 Application also discloses and claims another suction regulator that is less prone to generation of noise during operation since it includes a noise suppressor located in a portion of the tortuous passageway. The '890 Application claims priority from Provisional Application Ser. No. 62/829,731 filed on Apr. 5, 2019, entitled “System Including Suction Regulator For Automatically Removing Urine From A Female Patient And Method Of Use Of The System” and Application Ser. No. 62/872,397, filed on Jul. 10, 2019, entitled “System Including Sound Suppressed Suction Regulator For Automatically Removing Urine From A Female Patient Via An External Catheter And Method Of Removing Urine From A Female Patient Using An External Catheter”.

The subject invention is directed to a suction regulator and system including the same, which provides the advantages of the invention of the '980 Application in a structure that is simple is construction and low in cost and does not need to include any sound suppressor. Moreover, the subject invention provides a disposable integrated unit of a suction regulator and urine collecting canister for use between the female external catheter and a source of higher suction, e.g., a hospital's suction line. To that end the disposable integrated unit of this invention is designed in such a way that it allows far greater flow at low pressures than do the traditional wall regulators.

All of the references as cited herein are specifically incorporated by reference.

### SUMMARY OF THE INVENTION

One aspect of this invention is a system for automatically removing by suction urine voided by a patient comprising and external catheter, a receptacle, and a suction regulator. The external catheter is configured for external disposition in fluid communication with a urethra opening of the patient, whereupon urine voided by the patient is received by the external catheter. The receptacle or canister is configured for collecting urine and is configured to be coupled to a source of suction providing suction having a first value. The suction regulator has a regulated set-point value to which the suction regulator is fixedly set. The suction regulator is coupled to the receptacle or canister and the source of suction to regulate the amount of suction from the first value to the regulated set-point value. The regulated set-point value is lower than the first value, whereupon operation of the suction regulator applies regulated suction at the fixed regulated value to the external catheter to result in urine from the external catheter being carried through the suction regulator and into the receptacle or canister. The suction regulator comprises a regulated suction port configured to be coupled to the external catheter to apply regulated suction at the fixed regulated value to the external catheter, a line suction port configured to be coupled to the receptacle or canister and to the source of suction, a movable diaphragm, a valve seat configured to be engaged by a movable sealing member coupled to the movable diaphragm, and a passageway located at the valve seat and providing a straight fluid path to the line suction port.

In accordance with one preferred aspect of the system of this invention the receptacle or canister comprises a first port, a second port, and a hollow interior in fluid communication with the first and second ports. The first port is configured to be connected to the source of suction, and wherein the line suction port of the suction regulator is configured for coupling to the second port of the receptacle or canister. The suction regulator is configured to carry urine from the external catheter by the regulated suction through the suction regulator and into the second port of the receptacle or canister for collection in the hollow interior.

In accordance with another preferred aspect of the system of this invention the suction regulator additionally comprises a first chamber, a second chamber, and a biasing member. The first chamber is configured to have suction applied thereto from the canister. The second chamber is at atmospheric pressure. The movable diaphragm separates the first chamber from the second chamber, whereupon a differential pressure exists between the first and second chambers. The differential pressure imparts a differential pressure force on the movable diaphragm. The biasing member is configured to impart a counter force on the movable diaphragm that opposes the differential pressure force. The movable sealing member comprises a sealing disk coupled to the movable diaphragm.

In accordance with another preferred aspect of the system of this invention the valve seat and the movable sealing member are located in the first chamber. The valve seat surrounds an opening for fluids within the first chamber to flow therethrough into the passageway. The sealing member blocks the opening when the differential pressure force exceeds the counter force imparted by the biasing member.

In accordance with another preferred aspect of the system of this invention the suction regulator additionally comprises a housing in which the first and second chambers are located, and a hollow hub located in the first chamber. The valve seat and the passageway are located in the hub.

In accordance with another preferred aspect of the system of this invention the urine is carried through the suction regulator and into the receptacle or canister by air which is flowing at a high flow rate up to approximately 100 standard cubic feet per hour (SCFH)

In accordance with another preferred aspect of the system of this invention the regulated value of suction is within the range of approximately 40-80 mmHg.

In accordance with another preferred aspect of the system of this invention the second chamber is maintained at the atmospheric pressure by a bleed hole in the housing.

In accordance with another preferred aspect of the system of this invention the bleed hole is located within a recess in the housing. The recess has at least one opening at an end thereof to prevent blockage of the bleed hole.

In accordance with another preferred aspect of the system of this invention the suction regulator is mounted on the receptacle or canister.

In accordance with another preferred aspect of the system of this invention the system additionally comprises an adapter configured to be coupled between the source of suction and the receptacle or canister. The adapter comprises an inlet, a valve and a first connector. The inlet is configured to be connected to the source of suction. The first connector is configured to be connected to the receptacle or canister. The valve is configured to be in either a closed state or an open state, whereupon when in the closed state the receptacle or canister is isolated from the inlet, and when in the open state suction at the first value is provided from the inlet to the first connector and to the receptacle or canister.

In accordance with another preferred aspect of the system of this invention the adapter includes an internal passageway having one end in communication with the inlet and another end in communication with the first connector, and wherein the valve includes a rotatable portion having an opening extending therethrough, the rotatable portion intersecting the internal passageway, whereupon when the rotatable portion is in a first rotatable position the rotatable portion blocks the internal passageway between the inlet and the first connector, and when the rotatable portion is in a second position the opening unblocks the internal passageway between the inlet and the first connector to enable suction at the first value to appear at the first connector.

In accordance with another preferred aspect of the system of this invention the first connector is configured to have a first section of suction tubing having a first end connected to it and a second end connected to the receptacle or canister.

In accordance with another preferred of the system aspect of this invention the adapter additionally comprises a splitter including a second connector having a passageway section in fluid communication with the internal passageway between the inlet and the valve, whereupon suction at the first value is available from the inlet to the second connector irrespective of the state of the valve.

Another aspect of this invention is a system for automatically removing by suction urine voided by a patient comprising an external catheter, and an integrated suction regulator and urine collector unit. The external catheter is configured for external disposition in fluid communication with a urethra opening of the patient, whereupon urine voided by the patient is received by the external catheter. The integrated suction regulator and urine collector unit comprises a hollow receptacle or canister on which the suction regulator is mounted. The hollow receptacle or canister is configured for collecting urine. The suction regulator is configured for applying controlled suction to the external catheter from a source of suction having a first value. The suction regulator has a regulated set-point value to which the suction regulator is fixedly set and is operative regulate the amount of suction from the first value to the regulated set-point value. The regulated set-point value is lower than the first value, whereupon operation of the suction regulator applies regulated suction at the fixed regulated value to the external catheter to result in urine from the external catheter being carried through the suction regulator and into the receptacle or canister.

In accordance with one preferred aspect of the system of this invention, the urine is carried through the suction regulator and into the receptacle or canister by air which is flowing at a high flow rate up to approximately 100 standard cubic feet per hour (SCFH).

In accordance with another preferred aspect of the system of this invention the receptacle or canister comprises a first port and a hollow interior in fluid communication with the first port. The first port of the receptacle or canister is configured to be connected to the source of suction. The suction regulator comprises a first port and a second port. The second port of the suction regulator is connected to and in fluid communication with the hollow interior of the receptacle or canister and the source of suction. The suction regulator is configured for providing the regulated suction at the first port thereof. The first port of the suction regulator is configured to be coupled to the external catheter to carry urine from the external catheter by the regulated suction through the suction regulator and into the hollow interior of the receptacle or canister for collection therein.

In accordance with another preferred aspect of the system of this invention the receptacle or canister comprises a hollow body and a lid disposed over the hollow body, with the suction regulator being mounted on the lid.

In accordance with another preferred aspect of the system of this invention the regulated value of suction is within the range of approximately 40-80 mmHg.

In accordance with another preferred aspect of the system of this invention the suction regulator comprises a first chamber, a second chamber, a movable diaphragm and a biasing member. The first chamber is configured to have suction applied thereto from the receptacle or canister. The second chamber is at atmospheric pressure. The movable diaphragm separates the first chamber from the second chamber whereupon a differential pressure exists between the first and second chambers. The differential pressure imparts a differential pressure force on the movable diaphragm. The biasing member is configured to impart a counter force on the movable diaphragm that opposes the differential pressure force.

In accordance with another preferred aspect of the system of this invention the first chamber comprises a valve seat and a movable sealing member coupled to the movable diaphragm. The valve seat surrounds an opening for fluids within the first chamber to flow therethrough. The sealing member blocks the opening when the differential pressure force exceeds the counter force imparted by the biasing member.

In accordance with another preferred aspect of the system of this invention the second chamber is maintained at atmospheric pressure by a bleed hole in the housing located within a recess having at least one opening at an end thereof to prevent blockage of the bleed hole.

In accordance with another preferred aspect of the system of this invention the system additionally comprises an adapter configured to be coupled between the source of suction and the receptacle or canister. The adapter comprises an inlet, a valve and a first connector. The inlet is configured to be connected to the source of suction. The first connector is configured to be connected to the receptacle or canister. The valve is configured to be in either a closed state or an open state, whereupon when in the closed state the receptacle or canister is isolated from the inlet, and when in the open state suction at the first value is provided from the inlet to the first connector and to receptacle or canister.

In accordance with another preferred aspect of the system of this invention the adapter includes an internal passageway having one end in communication with the inlet and another end in communication with the first connector. The valve includes a rotatable portion having an opening extending therethrough. The rotatable portion intersects the internal passageway, whereupon when the rotatable portion is in a first rotatable position the rotatable portion blocks the internal passageway between the inlet and the first connector, and when the rotatable portion is in a second position the opening unblocks the internal passageway between the inlet and the first connector to enable suction at the first value to appear at the first connector.

In accordance with another preferred aspect of the system of this invention the first connector is configured to have a first section of suction tubing having a first end connected to it and a second end connected to the receptacle or canister.

In accordance with another preferred aspect of the system of this invention the adapter additionally comprises a splitter including a second connector having a passageway section in fluid communication with the internal passageway between the inlet and the valve, whereupon suction at the first value is available from the inlet to the second connector irrespective of the state of the valve.

Another aspect of this invention is a method for automatically removing by suction urine voided by a patient. The method entails providing an external catheter in fluid communication with a urethra opening of the patient, whereupon urine voided by the patient is received by the external catheter. A source of suction having a first value is provided. An integrated suction regulator and urine collector unit is also provided. It comprises a suction regulator and a receptacle or canister for collecting urine. The suction regulator has a regulated set-point value to which the suction regulator is fixedly set and which is lower than the first value. The integrated suction regulator and urine collector unit is coupled to the source of suction and operated to automatically regulate the amount of suction from the first value to the regulated set-point value and to apply suction at the regulated set-point value to the external catheter, whereupon urine from the external catheter is carried through the suction regulator and into the receptacle or canister.

In accordance with one preferred aspect of the method of this invention the urine is carried through the suction regulator by air which is flowing at a high flow rate up to approximately 100 standard cubic feet per hour (SCFH).

In accordance with another preferred aspect of the method of this invention the regulated value of suction is within the range of approximately 40-80 mmHg.

### DESCRIPTION OF THE DRAWING

FIG. 1 is an illustration of one exemplary system for automatically removing urine from a patient constructed in accordance with this invention;

FIG. 2 is an enlarged isometric view of one of the components, i.e., a suction regulator, forming a portion of the system of FIG. 1;

FIG. 3 is a bottom plan view of the suction regulator shown in FIG. 2;

FIG. 4 is an exploded isometric view of the components making up the suction regulator shown in FIG. 2;

FIG. 5 is an enlarged isometric view of one of the components, i.e., a cover, of the suction regulator shown in FIG. 2;

FIG. 6 is an enlarged isometric view of another of the components, i.e., a base, of the suction regulator shown in FIG. 2;

FIG. 7 is an enlarged isometric view, partially in vertical cross-section, of another of the components, i.e., a diaphragm, of the suction regulator shown in FIG. 2;

FIG. 8 is an enlarged side view of another of the components, i.e., a spring, of the suction regulator shown in FIG. 2;

FIG. 9 is an enlarged side view of another of the components, i.e., a suction tube, of the suction regulator shown in FIG. 2;

FIG. 10 is an enlarged sectional view taken along line 10-10 of FIG. 3.

FIG. 11 is an illustration of one exemplary system for automatically removing urine from a patient making use of an integrated suction regulator and urine collector unit constructed in accordance with this invention;

FIG. 12 is an enlarged vertical sectional view of integrated suction regulator and urine collector unit shown in FIG. 11;

FIG. 13 is an exploded isometric view of the components making up the integrated suction regulator and urine collector unit of FIG. 12;

FIG. 14 is an enlarged isometric view of a cap component of the up the integrated suction regulator and urine collector unit of FIG. 12;

FIG. 15 is an isometric view in transverse section of the cap component shown in FIG. 14;

FIG. 16 is an enlarged isometric view in transverse section of another component, i.e., a diaphragm, of the integrated suction regulator and urine collector unit of FIG. 12;

FIG. 17 is an enlarged side elevational view of another component, i.e., a biasing spring, of the integrated suction regulator and urine collector unit of FIG. 12;

FIG. 18 is an enlarged isometric view in transverse section of another component, a lid, of the integrated suction regulator and urine collector unit of FIG. 12;

FIG. 19 is an enlarged isometric view of another component, i.e., a float of an exemplary shut-off valve, of the integrated suction regulator and urine collector unit of FIG. 12;

FIG. 20 is an enlarged isometric view of another component, i.e., a basket for the float and which together with the float constitutes the exemplary shut-off valve, of the integrated suction regulator and urine collector unit of FIG. 12;

FIG. 21 is an enlarged vertical sectional view of the suction regulator portion of the integrated suction regulator and urine collector unit shown in FIG. 12.

FIG. 22 is an illustration of an adapter, like that of the '980 Application for connecting the systems of FIGS. 1 and 11 to a conventional threaded male wall connector of a hospital suction line;

FIG. 23 is an enlarged front view of the adapter shown in FIG. 22;

FIG. 24 is an end view of the adapter taken along line 24-24 of FIG. 23;

FIG. 25 is a longitudinal sectional view of the adapter taken along line 25-25 of FIG. 24;

FIG. 26 is an enlarged isometric view of a valve member forming a portion of the adapter shown in FIGS. 22-25;

FIG. 27 is an end view of the valve member shown in FIG. 26;

FIG. 28; is an illustration of an adapter/splitter, like that of the '980 Application, for connecting the systems of FIGS. 1 and 11 to a conventional threaded male wall connector of a hospital suction line;

FIG. 29 is an enlarged front view of the adapter and splitter shown in FIG. 28;

FIG. 30 is a side view of the adapter/splitter shown in FIG. 29;

FIG. 31 is an end view of the adapter/splitter taken along line 31-31 of FIG. 30;

FIG. 32 is a longitudinal sectional view of the adapter and splitter taken along line 32-32 of FIG. 31;

FIG. 33 is a side elevation view showing the adapter of FIGS. 22-27 being mounted on a conventional externally threaded connector of a port in the wall of a hospital or other care facility providing line suction; and

FIG. 34 is a side elevation view, similar to FIG. 33, but showing the adapter with splitter of FIGS. 28-32 being mounted on a conventional externally threaded connector of a port in the wall of a hospital or other care facility providing line suction and with a conventional suction regulator being mounted on the splitter.

### DETAILED DESCRIPTION OF PREFERRED EMBODIMENTS

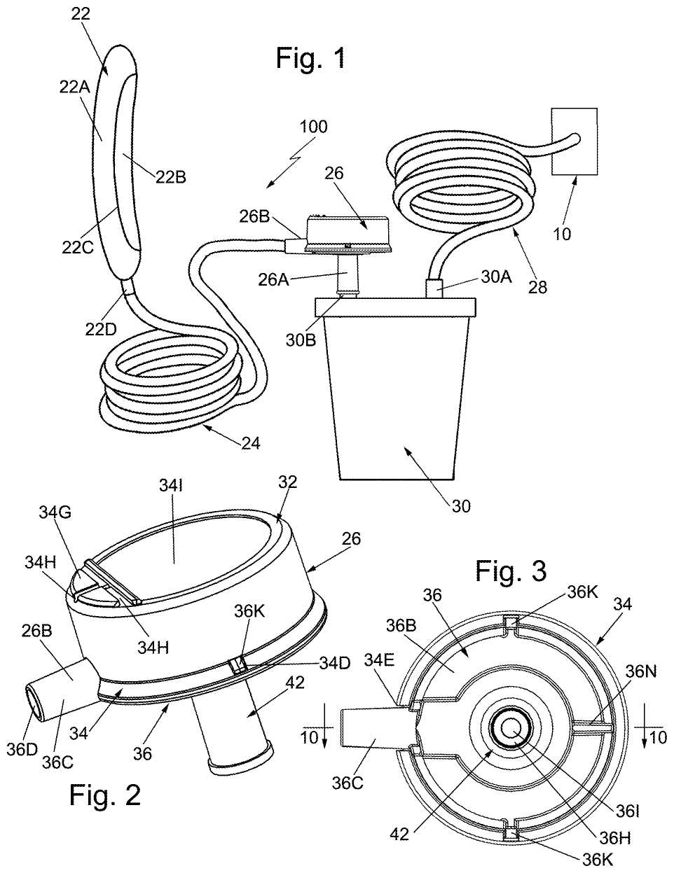

Referring now to the various figures of the drawing wherein like reference characters refer to like parts, there is shown in FIG. 1 one exemplary embodiment of a system 100 constructed in accordance with one exemplary preferred embodiment of this invention for automatically removing urine from a patient, e.g. a female. The details of the system 100 will be described later. Suffice it for now to state that the system 100 basically comprises an external catheter 22 which serves as a urine wicking member or device, a section 24 of conventional flexible suction tubing, a suction regulator 26, another section 28 of conventional flexible suction tubing, and a urine collection receptacle or canister 30. The urine wicking device can be constructed like any of the external catheters of the prior art described above. In the exemplary embodiment shown the device 22 is constructed like the PUREWICK® female external catheter.

The receptacle or canister 30 is of conventional construction and serves to automatically collect by suction urine removed from the patient by the external catheter 22. In particular, the receptacle or canister is a hollow cup-shaped member formed of any suitable material, e.g., a rigid preferably transparent plastic, such as polycarbonate. The reason for desired transparency of the plastic material is so that nurses and other attending personnel can assess hydration through urine coloration. In addition, they can readily detect the presence of blood in the urine, and calculate fluid balance by comparing fluid intake and urine output. To that end, the receptacle or canister may include volumetric lines printed on the outside of it to assist in the calculation of fluid balance.

The receptacle or canister 30 includes a port 30A (to be described later) that is configured to be connected to suction source, e.g., a wall regulator 10 of the hospital's main suction line. In particular, the distal end of the tubing section 28 is configured to be connected to the port 30A, while the proximal end of that tubing section is connected to the wall regulator 10. The wall regulator 10 should be set to line vacuum or the maximum available vacuum pressure if a line function is not available. The canister 30 includes another port 30B (to be described later) which is configured to be connected to a port 26A, referred to hereinafter as the “line suction port,” of the suction regulator 26. The suction regulator 26 will be described in detail later. Suffice it for now to state that it includes another port 26B, referred to hereinafter as the “regulated suction port,” which is connected to the proximal end of the tubing section 24. The regulated suction port 26B is connected to the proximal end of the tubing section 24. The distal end of the tubing section 24 is connected to the external catheter 22.

The suction regulator 26 is configured to enable flow through it from the external catheter to the canister nearing the maximum the hospital's suction line or regulator 10 is capable of sustaining without allowing the pressure to rise above a desired operating value, e.g., 40 mmHg, of the suction regulator 26 in the event the external catheter becomes sealed against the patient. With the preferred circuit of the system 100, i.e., with the suction regulator 26 located between the external catheter 22 and the urine collecting canister 30, the regulator 26 will be closer to the catheter 22 than if it was located between the canister 30 and the hospital suction line or regulator 10, thereby enabling the maximum possible urine flow, but necessitates the urine flowing through the regulator. To that end, the suction regulator 26 is intended to be a non-sterile, single-patient-use disposable unit having a fixed (e.g., factory-established) regulated set-point value to be described later.

The external catheter 22 basically comprises a soft, elongated hollow flexible member 22A in which a body of soft gauze 22B is located. The member 22A includes a longitudinally extending side window or opening 22C exposing the soft gauze body. The external catheter is designed to be disposed between the woman patient's separated gluteus and labia and in fluid communication with her urethral opening. A suction port 22D is located at one end of the member 22A and in fluid communication with the gauze body. The suction port is configured to be connected to the distal end of the tubing section 24, whereupon suction will be applied by that tubing section to the interior of the member 22A. As such any urine which the female patient had voided into the gauze body 22B will be pulled into the suction tubing 24 and from there to the suction regulator 26. In a typical application the length of the tubing section 28 connecting the receptacle or canister 30 to the wall regulator 10 is approximately 6 feet, with the inner diameter of the passageway through the tubing section 28 being approximately 0.25 inch. The length of the tubing section 24 connected between the regulated suction port 26A of the suction regulator 26 and the external catheter in a typical application is approximately 6 feet with the inner diameter of the passageway through the tubing section 24 being approximately 0.25 inch.

With the system 20 as just described when suction is applied to the system 20 from the hospital's suction line or wall regulator 10, that high level of suction is conveyed through the canister to the line suction port 26A of the suction regulator 26, whereupon it is regulated (e.g., reduced) by operation of the suction regulator to a much lower operating level, e.g., 40 mmHg. That reduced or regulated suction will appear on the regulated suction port 26B of the regulator and from there through the associated tubing section 24 to the external catheter 22 to thereby draw urine from the external catheter back through the tubing section 24 into and through the regulator 26, and into the receptacle or canister 30 for collection therein.

It should be noted that for many applications the operating level is preferably approximately 40 mmHg. However, that level could be raised up to approximately 80 mmHg, since some hospitals are comfortable with higher vacuum pressures. As will be appreciated by those skilled in the art the higher pressure makes the height of the receptacle or canister 30 relative to the patient less important.

If desired the system 100 may also include an overflow detector of any suitable construction to provide an indication that the amount of urine within receptacle has reached a predetermined threshold, e.g., is about to overflow, and/or to provide a signal to a controller (not shown) stop to halt the operation of the system so that no further urine is drawn into the receptacle until it can be emptied. For example, the canister 30 may include a shut off float valve and/or a filter at the port 30A to prevent possible contamination of the hospital's main suction. Including a filter can create a pressure drop across the filter. In such circumstances it is advantageous to have the suction regulator 26 positioned upstream of the filter in order to maximize airflow rate while maintaining a low safe suction set-point.

The suction regulator 26 serves to ensure that a desired level of suction is applied to the external catheter to ensure proper and safe operation of the system, i.e., to maximize the rate at which urine may be withdrawn from the catheter into the receptacle or canister without subjecting the delicate tissue of the woman at her urethral opening to injury, e.g., a hematoma, from excess suction thereat.

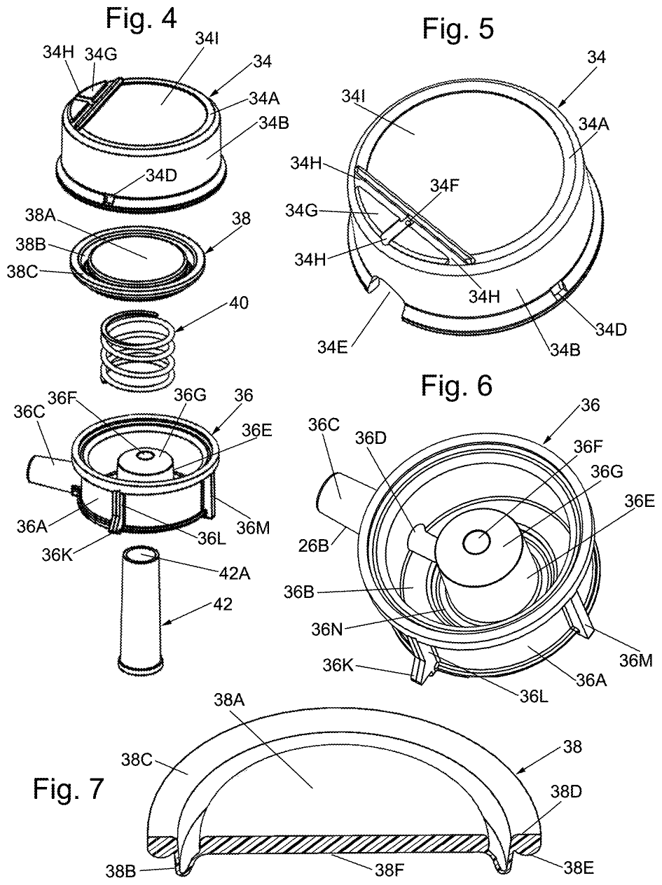

Turning now to FIGS. 2 and 4 the components making up the suction regulator 26 are shown. In particular, the suction regulator includes a housing assembly 32 (FIG. 2) composed of a lid or cover 34 (FIG. 4) and a base 36 (FIG. 4). The base 34 and the cover 36 are formed of any suitable material, e.g., Acrylonitrile Butadiene Styrene (“ABS”), and are configured to be connected together, as will be described later, to form the hollow housing assembly 32 enclosing other interior components making up the suction regulator 26. Those interior components are a flexible diaphragm 38 and a helical compression spring 40. The suction regulator also includes a suction tube 42 that is mounted on the base 34 of the housing assembly and which forms the heretofore identified line suction port 26A.

The cover or lid 34 is a generally cup shaped member having a generally planar top wall 34A and a downwardly projecting circular sidewall 34B. The sidewall is configured to be secured to a circular sidewall 36A of the base 36 to form a hollow interior in which two chambers (a lower chamber 26C and an upper chamber 26D, to be described later) of the suction regulator 26 are located. To that end, as best seen in FIGS. 3, 4, 6 and 7 the base 34 is also a generally cup shaped member having the heretofore identified circular sidewall 36A and a generally planar bottom wall 36B. The sidewall 34A projects upward from the bottom wall 34B and extends about a central axis X of the suction regulator. A tubular extension 36C extends generally parallel to the undersurface of the bottom wall 36B and radially outward from the axis X. The tubular extension 36C forms the heretofore identified regulated suction port 26B and includes a passageway 36D extending through it.

A hollow cup-shaped projection or hub 36E extends upward from the bottom wall 36B and is centered on the axis X. The upper end of the projection or hub 36E includes a circular opening 36F centered on the axis X, with the upper end portion of the projection or hub contiguous with the circular opening being a beveled or conical surface. The portion of the beveled surface contiguous with the opening 36F forms a valve seat 36G for the suction regulator. The valve seat is configured to be engaged by a sealing disk forming a portion of the diaphragm 38 (as will be described later). A tubular extension 36H extends downward from the opening 36F and includes a central passageway 36I extending the length of the extension from the opening 36F to the bottom of the extension. The extension 36H is configured to be received and held within a passageway extending through the suction tube 42 to secure the suction tube to the housing assembly 32.

As best seen in FIGS. 6 and 10, the upper edge of the sidewall 36A is in the form of an annular recess or ledge 36J on which a peripheral portion of the diaphragm 38 is disposed. The disposition of the peripheral portion of the diaphragm on the annular recess or ledge 36J of the base 36 results in the formation of the lower chamber 26C of the suction regulator. The lower chamber is bounded by the inner surface of the circular sidewall 36A, the undersurface of the diaphragm 38, the outer surface of the projection or hub 36E, and the portion of the top wall 36B between the circular sidewall 36A and the projection or hub 36E. The inner end of the passageway 36D is in fluid communication with the lower chamber 26C. The upper chamber 26D of the suction regulator is bounded by the upper surface of the diaphragm 38, the inner surface of the top wall 34A and a low height annular ridge 34C (FIG. 10) projecting downward from the inner surface of the top wall 34A.

The sidewall 34A of the cover 34 includes a pair of diametrically opposed notches 34D immediately adjacent the flared lower edge of the sidewall. The notches are configured to receive respective diametrically opposed projecting tabs 36K of the base to secure the cover to the base and thus complete the housing assembly 32. Each of the tabs is located at the bottom of an elongated strut 36L (FIGS. 4 and 6). The struts reinforce the sidewall 36A. Another elongated reinforcing strut 36M is located between the struts 36L. The sidewall 34B of the lid or cover also includes an arcuate recess 34E (FIGS. 2 and 5) in the lower edge of the sidewall located midway between the notches 34D. The recess 34E serves to receive the tubular extension 36C of the base 34 when the lid or cover is secured to the base.

The diaphragm 38 is a rolling diaphragm, which is best seen in FIGS. 4 and 7. It is a circular member that is formed of any resilient flexible material, e.g., silicone. The diaphragm includes a generally planar relatively thick, e.g., 0.1 inch, circular central portion 38A, a generally U-shaped annular edge portion 38B surrounding the central portion and terminating in a flanged generally planar thickened periphery 38C. The thickened periphery of the diaphragm has a generally planar top surface 38D and an arcuate undersurface 38E. The undersurface 38E is configured to rest on the annular recess or ledge 36J of the sidewall 36A of the base. To that end, the ledge is correspondingly shaped to the arcuate undersurface 38E. The undersurface of the central portion 38A of the diaphragm forms a valve element in the form of a sealing member or sealing disk 38F, that is configured to be brought into engagement with the valve seat 36G. In particular, the sealing disk 38F of the diaphragm is disposed over the valve seat 36G, with the thickened periphery 38C of the diaphragm disposed on an annular ledge 36J at the upper end of the sidewall 36A between that ledge and the undersurface of the planar top wall 34A of the cover.

With the cover secured to the base as described above, the thickened periphery of the diaphragm is tightly sandwiched between the ledge 36J and the inner surface of the top wall 34A of the cover and confined within the bounds of the annular ridge 34C projecting downward from the inner surface of that top wall. This arrangement divides the interior of the suction regulator into the two heretofore identified chambers, namely, the lower chamber 26C and the upper chamber 26D (FIG. 10).

The cover or lid 34 also includes a small bleed or vent 34F to the ambient atmosphere which will be referred to as the atmospheric reference port (FIGS. 5 and 10). The atmospheric reference port ensures that the upper chamber 26D will be at the pressure of the ambient atmosphere. In particular, the port 34F extends through the thickness of the cover and is in fluid communication with the interior of upper chamber 26D to maintain that chamber at atmospheric pressure. Inasmuch as the atmospheric reference port 34F is located in the top surface of the cover, it is susceptible to being blocked or covered by a sticker, some other object or even the finger of a user. To prevent such an occurrence the lid or cover is shaped to prevent blockage of the port 34F. In particular, the lid or cover includes a thickened portion 34G located adjacent the port 34F with an elongated shallow tripartite or T-shaped recess or slot 34H extending into the thickened portion. The outer or top end of the atmospheric reference port 34F is located at the bottom of the slot 34H at the intersection of the slot's various three sections and is in fluid communication with each of those sections. The outer end of each of the slot sections is open. Thus, if something should be on the top surface of the thickened portion 34G of the lid or cover disposed over the atmospheric reference port 34F air can still enter into that port via any open end of the T-shaped slot 34H.

A label (not shown) bearing indicia or information regarding the suction regulator 26 may be fixedly secured within a very shallow recess 34I (FIGS. 5 and 10) in the top surface of the lid or cover adjacent the thickened portion 34G so its presence does not block the T-shaped slot 34H.

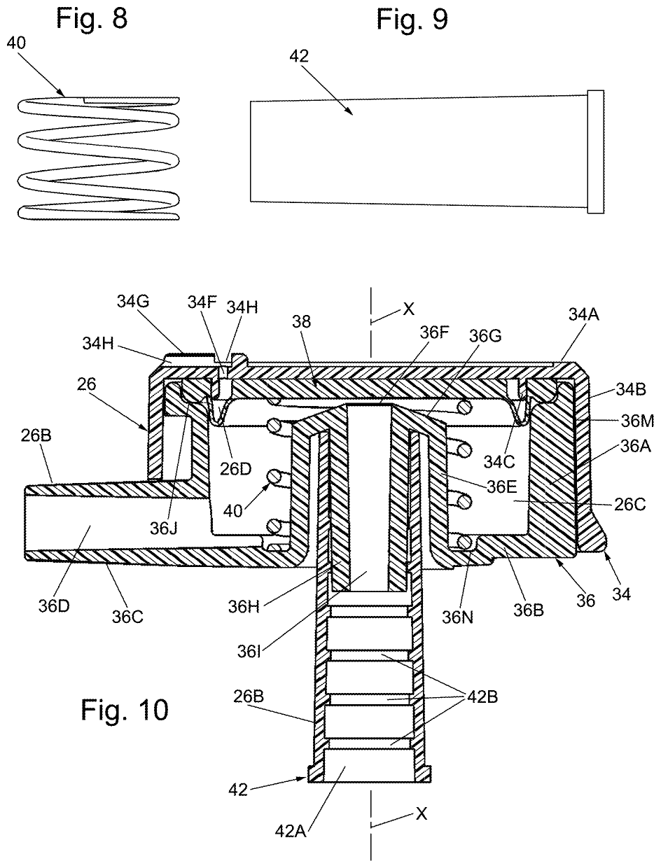

Turning now to FIGS. 4, 8 and 10, the details of the spring 40 will now be described. As can be seen the spring 40 is a helical compression spring and is formed of any suitable material, e.g., stainless steel. The spring is located within the lower chamber 26C. In particular, the upper end of the spring is in engagement with the undersurface 38F of the central portion 38A of the diaphragm and surrounding the projection or hub 36E, and with the lower end of the spring located within an annular recess 36N in the bottom wall 36B of the base. The spring 40 is under compression to bias the diaphragm 38 upward.

As mentioned above, the suction regulator 26 regulates the level of suction to a desired operating value, e.g., 40 mmHg, and provides the regulated suction to the external catheter (the urine wicking member) 22. To that end, the regulator 26 is configured to limit the amount of suction applied to the external catheter to that desired value even if a level of suction greater than that predetermined value is applied to the suction regulator from the suction source (particularly if the suction source is at a much higher level, which will typically be the case if the suction source is the hospital's suction line). The predetermined or desired suction value (hereinafter referred to has the “regulator's set-point” or “regulated set-point value”) is fixed and is factory-established by the dimensions of the lower chamber 26C, the spring 40 and the stiffness of the diaphragm 38 and in particular its central portion 38A.

As should be appreciated by those skilled in the art, the pressure within the upper chamber 26D will be equal to atmospheric pressure by virtue of the communication of that chamber with the ambient atmosphere via the atmospheric reference port 34F. Thus, when suction is applied from the suction source 10, the pressure within the lower chamber 26C will be lower than the atmospheric pressure within the upper chamber 26D. The differential pressure between the upper and lower chambers will force the diaphragm 38 downward toward the valve seat 36G. The spring 40, however, will impart a counter force on the diaphragm that opposes the force pushing the central portion of the diaphragm downward such that the level of suction appearing at the regulated suction port 26B is the desired operating value, e.g., 40 mmHg.

If the suction applied via line suction port 26A is greater that the predetermined value or level the central portion of the diaphragm 38 will move such that its sealing disk portion 38F comes into engagement with the valve seat 36G, thereby isolating the lower chamber 26C from the suction appearing on the line suction port 26A. This action thereby limits the level of suction in lower chamber and ultimately at external catheter 22 to the predetermined level (operating value). If, however, the suction applied via line suction port is less than the predetermined operating level the diaphragm will only move part of the way downward. As such the level of suction applied to the line suction port 26A will equal that in the regulated suction port 26B and that applied to the external catheter 22.

Turning now to FIGS. 4, 9 and 10 the details of the suction tube 42 will now be described. As can be seen the suction tube is a tubular member, which is formed of any suitable material, e.g., polyvinylchloride (“PVC”). It includes a central passageway 42A, extending through it. A plurality of annular ridges 42B is equidistantly spaced from one another along the length of the passageway 42A. The annular ridges form engaging surfaces which are configured to frictionally engage respective portions of the tubular extension 36H to connect the suction tube 42 to the suction regulator 26.

In accordance with one exemplary preferred embodiment of the suction regulator 26, inner diameter of the lower chamber 26C is approximately 1.5 inch. The inner diameter of the upper chamber is approximately 1.5 inch. The spring is configured to naturally apply a bias force of approximately 1.0 pound. The inner diameter of the passageway 361 and hence the valve seat 36G is approximately 0.25 inch. The inner diameter of the passageway 36D is approximately 0.25 inch. The atmospheric reference port 34F is approximately 0.035 inch in diameter. Each tubing section 24 and 28 is conventional having an internal passageway of approximately 0.25 inch in diameter, and each section is approximately six feet in length, but could be shorter or longer depending upon the application.

With a suction regulator 226 sized as just described, in a system like that described during typical operation the flow rate of air can be up to 100 SCFH as compared to the 15 SCFH rate observed with some commercially available wall regulator set to the suggested 40 mmHg. The additional flow allows for increased urine capture at the interface of the actual catheter, faster drying of the catheter (which helps prevent skin breakdown and infection) and pulls the urine through the tubing into the canister 222 more efficiently. This is especially true if the tubing drapes down below the height of the patient and canister.

As should be appreciated by those skilled in the art since the suction regulator 26 has a fixed regulated set-point value, users of the system 100 do not have to pick and set a particular value for the suction to be applied to the patient's external catheter 22. As such a safe level of suction will be automatically be applied to the patient by the suction regulator 26 without requiring hospital personnel or other operators to set the desired value of suction to be applied by the external catheter. Moreover, since the suction regulator 26 has a fixed regulated set-point value, there is no need for it to include any dials or other indicators to provide the hospital personnel or other operators with a reading of the suction level being applied so that they could set the regulator to the desired set-point value. Accordingly, suction regulators constructed in accordance with this invention can be simple in construction, low in cost and easy to use. Moreover, the flow path of air through suction regulators constructed in accordance with this invention is less tortuous, e.g., there are less right angle turns, than the suction regulators disclosed in our aforementioned copending provisional applications, so that the suction regulators of this invention produce less noise during operation, and do not need to include any sound suppressors.

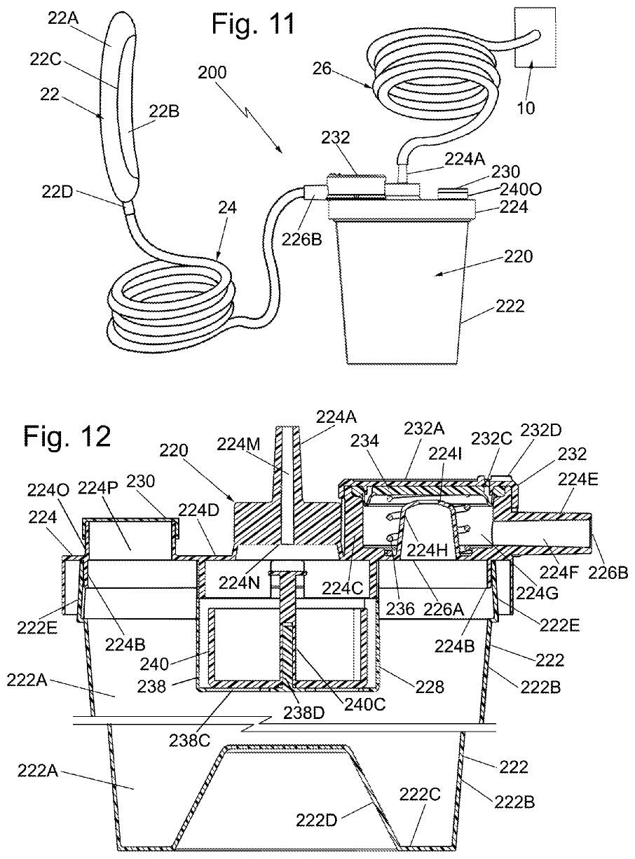

Turning now to FIG. 11 there is shown another exemplary embodiment of a system 200 constructed in accordance with one exemplary preferred embodiment of this invention for automatically removing urine from a patient, e.g., a female. The system 200 basically comprises the external female catheter 22, the section 24 of conventional flexible suction tubing, an integrated suction regulator and urine collector unit (also referred to hereinafter as an integrated urine collection canister and suction regulator unit) 220, and another section 26 of conventional flexible suction tubing 26. The external female catheter can be constructed like any of the external catheters of the prior art described above. In the exemplary embodiment shown in FIG. 11 it is constructed like the PUREWICK® female external catheter, whereupon the details of its construction and operation will not be reiterated in the interest of brevity.

The external female catheter 22 is designed to be disposed between the woman patient's separated gluteus and labia and in fluid communication with her urethral opening. A suction port 22D is located at one end of the member 22A and in fluid communication with the gauze body. The suction port is configured to be connected to the distal end of the tubing section 24 from which suction will be applied to the external catheter to cause urine to be drawn into the integrated suction regulator and urine collector unit 220 for collection therein. That unit will be described in considerable detail later. Suffice it for now to state that the unit 220 includes a hollow receptacle or canister 222 having a lid 224 including a built-in suction regulator 226. The suction regulator 226 includes a first internal port 226A (FIG. 12) and a second external port 226B. The external port 226B (hereinafter referred to as the “regulated suction port”) is configured to be connected to the proximal end of the tubing section 24. The distal end of the tubing section 24 is connected to external catheter 22. The internal port 226B is in fluid communication with the hollow interior 222A of the canister 222. The unit 220 includes another external port 224A which is in fluid communication with the hollow interior of the canister 222 via an exemplary isolation valve 228, each of which will be described later. As will be described later the isolation valve is normally open, but closes, to close or seal the port 224A when the urine collected in the canister 222 reaches the level of the isolation valve and thereby prevents any urine in the canister from entering into that port. The port 224A is configured to be connected to the distal end of the tubing section 26. The proximal end of the tubing section 26 is connected to a source of suction, e.g., a wall regulator or valve 10 of the hospital's main suction line so that suction will be applied from the source 10 through the tubing section 26 and port 224A into the interior of the canister 222. Since the internal port 226A of the suction regulator is in fluid communication with the hollow interior 222A of the canister 222 via the normally open isolation valve 228, the suction produced by the source 10 will be applied to the port 226A. As such the port 226A will be referred to hereinafter as the “line suction port”.

The receptacle or canister 222 of the unit 220 serves as the means for collecting urine removed from the patient by the external catheter 22. In particular, when suction is applied to the system 200 from the hospital's suction line or wall regulator 10, that high level of suction is conveyed through the tubing section 26 to the port 224A from whence it is conveyed to the hollow interior of the canister 222 to the line suction port 226A of the suction regulator 226, whereupon the suction regulator automatically regulates it to the desired regulated (e.g., reduced) operating value like that described with reference to the system 100. Like system 100, the suction regulator 226 of the system 200 limits the amount of suction applied to the external catheter to regulated set point value even if a level of suction greater than that predetermined value is applied to the suction regulator from the suction source (particularly if the suction source is at a much higher level, which will typically be the case if the suction source is the hospital's suction line).

The reduced or regulated suction at the regulated set-point value will appear on the regulated suction port 226B of the regulator and from there through the associated tubing section 24 to the external catheter 22 to thereby draw urine from the external catheter back through the tubing section 24 and through the normally open shut-off valve 228 into the hollow interior 222A of the canister 222 for collection therein.

It should be noted that for many applications the set-point of the regulator 226 is preferably approximately 40 mmHg. However, that level could be raised up to approximately 80 mmHg, by appropriate selection and sizing of the suction regulator's components. The higher pressure makes the height of the integrated suction regulator and urine collector unit 220 relative to the patient less important.

The suction regulator 226 serves to ensure that the desired set-point value of suction is applied to the external catheter 22 to ensure proper and safe operation of the system 200, i.e., to maximize the rate at which urine may be withdrawn from the catheter into the receptacle or canister 222 without subjecting the delicate tissue of the woman at her urethral opening to injury, e.g., a hematoma, from excess suction thereat. To that end, the suction regulator 226 is configured to enable air to flow through it from the external catheter 22 to the canister 222 nearing the maximum the hospital's suction line or regulator 10 is capable of sustaining without allowing the pressure to rise above the regulator's set-point, e.g., 40 mmHg, in the event the external catheter 22 becomes sealed against the patient. Since the suction regulator 226 is located between the external catheter 22 and the urine collecting canister 222, the regulator 226 will be closer to the catheter 22 than if it was located between the canister 222 and the hospital suction line or regulator 10, thereby enabling the maximum possible urine flow, but such an arrangement necessitates the urine flowing through the suction regulator. Thus, in accordance with one preferred aspect of this invention the integrated urine collection canister and suction regulator unit 220 is intended to be a non-sterile, single-patient-use disposable unit.

As will be described later the lid 224 of the unit 220 includes a spout which is normally closed by a removable cap 230. With the cap removed the collected urine within the canister 222 can be poured out of the canister through the spout, and after the canister emptied, the removable cap 230 replaced so that the unit 220 can be reused with that patient. The use of a spout to empty the urine contents from the canister is optional, so that the integrated urine collection canister and suction regulator unit 220 may be constructed without any spout or means to empty the urine from the canister. In any case, whether the integrated urine collection canister and suction regulator unit 220 includes a spout or other means to empty its urine contents or not, the unit 220 is designed for single-patient-use. Thus, when that patient no longer needs to use an external catheter, the unit 220 is discarded.

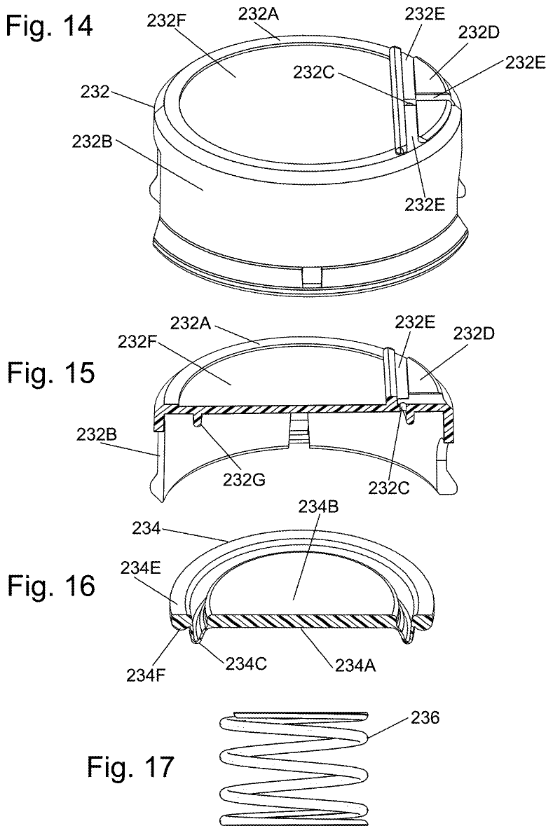

Turning now to FIGS. 12-18, the construction of the integrated urine collection canister and suction regulator unit 220 will now be described. As noted earlier it includes the hollow receptacle or canister 222 and the lid 224. The canister or receptacle 222 is a hollow cup-shaped member formed of any suitable material, e.g., a rigid preferably transparent plastic, such as polycarbonate, and having a side wall 228. The reason for desired transparency of the plastic material for the canister or receptacle is so that nurses and other attending personnel can assess hydration through urine coloration. In addition, they can readily detect the presence of blood in the urine, and calculate fluid balance by comparing fluid intake and urine output. To that end, the receptacle or canister may include volumetric lines printed on the outside of it to assist in the calculation of fluid balance.

The lid 224 is formed of any suitable material, e.g., a rigid plastic, such as ABS, and includes a generally planar top wall 230, a peripheral downwardly projecting somewhat tapering sidewall 222B and a generally planar bottom wall 222C having a central recess 222D. The upper edge 222E of the sidewall 222B of the canister 222 is secured to a downwardly projecting circular sidewall 224B of the lid to mount the canister on the lid. The lid is preferably a unitary body formed, e.g., molded, of a rigid plastic, such as ABS.

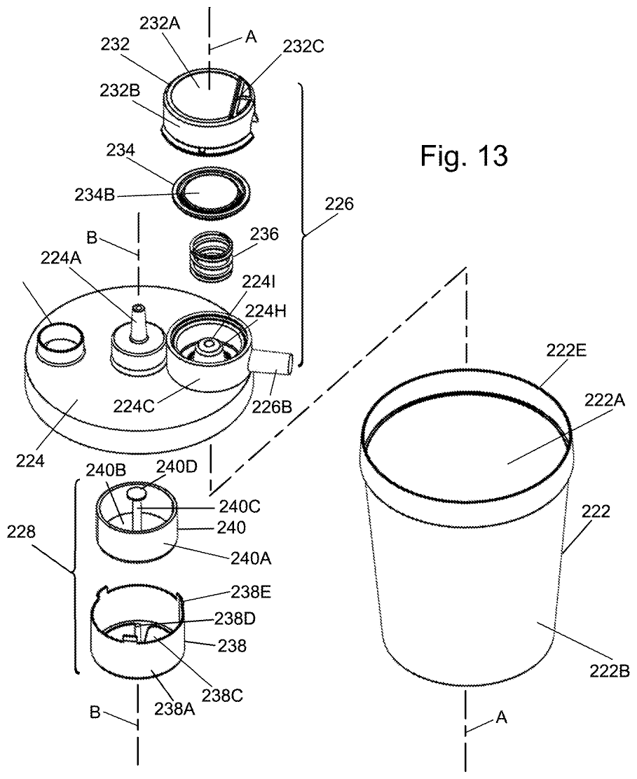

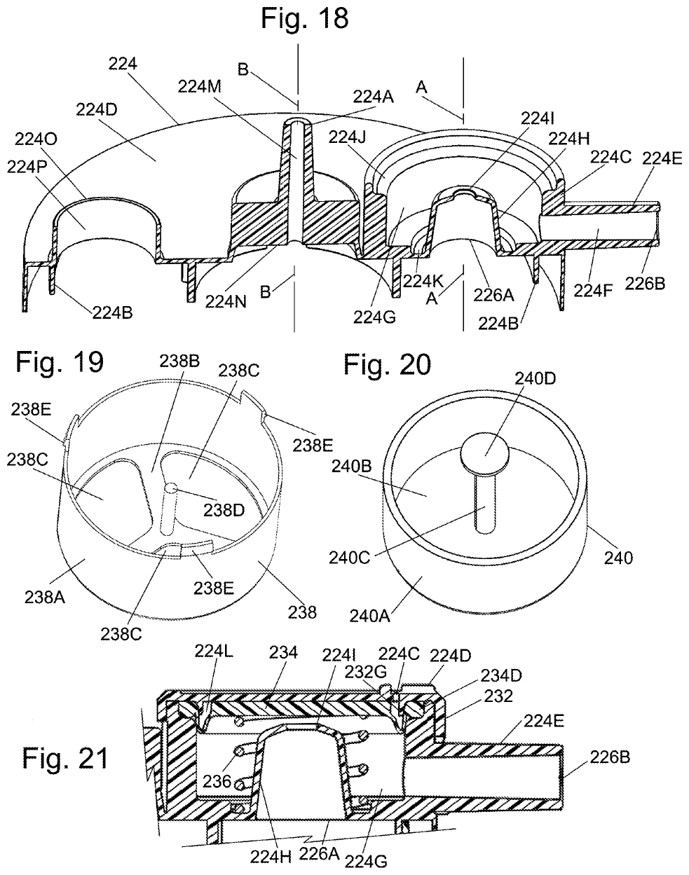

The components making up the suction regulator 226 are located within a housing section that is composed of a cover 232 and an upwardly projecting circular sidewall 224C (FIG. 18) of the lid. The cover 232 is best seen in FIGS. 13-15 and is a generally cup-shaped member having a generally planar top wall 232A and a circular sidewall 232B projecting downward from the top wall. The cover is configured to be fixedly mounted onto the sidewall 224C of the lid after the components making up the suction regulator 226 are in place and assembled. Those components are a flexible diaphragm 234 having an undersurface which forms a sealing disk 234, and a helical compression spring 236. The circular sidewall 224C projects upward from the top wall 224D of the lid and is centered about an axis A of the suction regulator 226. The downwardly projecting circular sidewall 232B of cover is configured to be fixedly secured, e.g., snap-fit, to the upwardly projecting circular sidewall 224C of the lid to form a hollow interior in which two chambers (an upper chamber 224L and a lower chamber 224G to be described later) of the suction regulator 226 are located.

A tubular extension 224E extends radially outward from the sidewall 224C and generally parallel to the top wall 224D. The extension 224E forms the heretofore identified regulated suction port 226B. To that end it includes a passageway 224F extending through it. The passageway 224F extends radially from the axis A and has an outer or free end which is open. Thus, that the distal end of the flexible tubing section 24 can be connected to the tubular extension 224E whereupon the passageway extending through that tubing section will be in fluid communication with the passageway 224F. The opposite end of the passageway 224F terminates at an opening in the sidewall 224E, which is in fluid communication with a lower chamber 224G. A portion 256 of the top wall 224D of the lid forms the bottom wall of the lower chamber 224G. A hollow cup-shaped projection or hub 224H extends upward from the wall 224D in the lower chamber 224G and is centered on the axis A. The upper end of the projection 224H includes a circular opening centered on the axis A, with the upper end portion of the projection contiguous with the circular opening being a beveled or conical surface and forming a valve seat 224I for the suction regulator. The valve seat is configured to be engaged by a sealing disk forming a portion of the diaphragm, as will be described later. The opposite or lower end of the projection or hub 224H is also open and forms the heretofore identified interior port 226A. Thus, the interior port 226 is in fluid communication with both the interior of the receptacle or canister 222 and the interior of the lower chamber 224G of the suction regulator. The lower chamber 224G is defined by the inner surfaces of the circular sidewall 224C, the undersurface of the diaphragm 240, the outer surface of the cup-shaped projection or hub 224H, and the portion of the top wall portion 224D between the circular sidewall 224C and the cup-shaped projection 224H.

The diaphragm 234 constitutes a rolling diaphragm, which is best seen in FIG. 15. It is a circular member that is formed of any resilient flexible material, e.g., Nitrile. The diaphragm includes a generally planar circular central portion 234B, a generally U-shaped annular edge portion 234C surrounding the central portion and terminating in a flanged generally planar thickened periphery 234D. The undersurface of the central portion 234B of the diaphragm forms the heretofore identified sealing disk. That disk serves as the heretofore mentioned valve element, and is configured to be brought into engagement with the valve seat 224I. The thickened periphery of the diaphragm has a generally planar top surface 234E and an arcuate undersurface 234F. The undersurface 234F is configured to rest on a correspondingly shaped annular ledge 224J of the sidewall 224C of the lid. The central portion 234B of the diaphragm is disposed over the valve seat 224I, with the thickened periphery 234D of the diaphragm disposed on an annular ledge 224J at the upper end of the sidewall 224C between that ledge and the undersurface of the planar top wall 232A of the cover. With the cover 232 secured to the annular sidewall 248 of the lid 224 the thickened periphery of the diaphragm is tightly sandwiched between the ledge 224J and the inner surface of the cover and confined within the bounds of an annular ridge 232G projecting downward from the inner surface of the cover. This arrangement divides the interior of the suction regulator into the two chambers, namely, the heretofore identified lower chamber 224G and the upper chamber 224L (FIG. 21). As best seen in FIG. 21 the upper chamber 224L is formed between the inner surface of the upper wall of the cup-shaped housing section and the upper surface of the diaphragm 234. A passageway 224M extends through the line suction port 224A and terminates at its bottom in a planar surface 224N. The portion of the surface 224N contiguous with the bottom of the passageway 224M is configured to be engaged by a float (to be described later) of the exemplary shut-off valve 228 to prevent urine from gaining ingress into the passageway 224M.

The top wall 232A of the cup-shaped cover includes a small opening or vent 232C (FIGS. 4 and 5) which is in fluid communication with the ambient atmosphere and which will be referred to as the atmospheric reference port 232C. The atmospheric reference port 232C ensures that the upper chamber will always be at the pressure of the ambient atmosphere. In particular, the port 232C extends through the thickness of the top wall of the cover and is in fluid communication with the interior of upper chamber to maintain that chamber at atmospheric pressure. Inasmuch as the atmospheric reference port 232C is located in the top surface of the cover, it is susceptible to being blocked or covered by a sticker, some other object or even the finger of a user. To prevent such a blockage occurrence the upper surface of the top wall 236 of the cover is shaped to prevent blockage of the atmospheric reference port. In particular, the top wall of the cover includes a thickened portion 323D having a generally T-shaped slot or groove 232E therein. The atmospheric reference port 232C is located at the bottom of the T-shaped slot or groove 232E at the intersection of the slot's three sections and is in fluid communication with each of those sections. The outer end of each of the slot sections is open. Thus, if something should be on the top surface of the thickened portion 232D of the cover and disposed over the atmospheric reference port 232C air can still enter into that port via any open end of the T-shaped slot 232E. If desired a label (not shown) bearing indicia or information regarding the suction regulator 226 can be fixedly secured to the cover. To that end the top wall of the cover includes a shallow recess 232F adjacent the thickened portion 232D for receipt of the label. Thus, when the label is disposed and secured within the shallow recess its presence will not block the T-shaped slot 232E.

Turning now to FIGS. 12, 13 and 17, the details of the spring 236 will now be described. As can be seen the spring 236 is a helical compression spring and is formed of any suitable material, e.g., stainless steel. The spring is located within the lower chamber 224G. In particular, the upper end of the spring is in engagement with the undersurface of the central portion 234A of the diaphragm and surrounding the projection or hub 224H, and with the lower end of the spring located within an annular recess 224K in the bottom wall of the lower chamber 224G. The spring 236 is under compression to bias the diaphragm 234 upward.

As mentioned above, the suction regulator 226 regulates the level of suction to a desired operating value, e.g., 40 mmHg, and provides the regulated suction to the external catheter (the urine wicking member) 22. To that end, the regulator 226 is configured to limit the amount of suction applied to the external catheter to that desired value even if a level of suction greater than that predetermined value is applied to the suction regulator from the suction source (particularly if the suction source is at a much higher level, which will typically be the case if the suction source is the hospital's suction line). The predetermined or desired suction value (hereinafter referred to has the “regulator's set-point” or “regulated set-point value”) is fixed and is factory-established by the dimensions of the lower chamber, the spring 244 and the stiffness of the diaphragm and in particular its sealing disk portion.

As should be appreciated by those skilled in the art, the pressure within the upper chamber will be equal to atmospheric pressure by virtue of the communication of that chamber with the ambient atmosphere via the atmospheric reference port 232C. Thus, when suction is applied from the suction source 10, the pressure within the lower chamber 224G will be lower than the atmospheric pressure within the upper chamber. The differential pressure between the upper and lower chambers will force the diaphragm 232 downward toward the valve seat 224I. The spring 236, however, will impart a counter force on the diaphragm that opposes the force pushing the central portion of the diaphragm downward such that the level of suction appearing at the regulated suction port 226B is the desired operating value, e.g., 40 mmHg.

If the suction applied via line suction port 224A is greater than the predetermined value or level, the central portion of the diaphragm 240 will move such that its sealing disk comes into engagement with the valve seat 224I thereby isolating the lower chamber 224G from the suction appearing on the line suction port 224A. This action thereby limits the level of suction in lower chamber and ultimately at external catheter 22 to the predetermined level (operating value). If, however, the suction applied via line suction port is less than the predetermined operating level the diaphragm will only move part of the way downward. As such the level of suction applied to the line suction port 224A will equal that in the regulated suction port 226B and that applied to the external catheter 22.

As mentioned earlier the integrated urine collection canister and suction regulator unit 220 includes an isolation valve 228. That valve basically comprises a basket 238 and a float 240. As best seen in FIGS. 13 and 19 the basket 238 is a cup shaped member having a circular sidewall 238A and a generally planar bottom wall 238B. The bottom wall includes three wedge or pie-shaped openings 238C equidistantly spaced about the central axis B of the unit 220. A cylindrical post 238D projects upward from the inner surface of the bottom wall 238B and is centered on the axis B. Three flexible fingers or tabs 238E project upward from the top edge of the sidewall 238A and are equidistantly spaced from one another about the axis B. Each finger or tab includes a flanged free end which arranged to snap-fit into a respective opening or slot (not shown) in the lid 224 to mount the basket on the underside of the lid.

The float 240 is located within the basket and is best seen in FIGS. 13 and 20. The float is also a cup-shaped member having a circular sidewall 240A and a generally planar bottom wall 240B. The sidewall 240A is slightly smaller in outside diameter than the inside diameter of the basket's sidewall 238A so that it can fit therein. A cylindrical post 240C projects upward from the inner surface of the bottom wall 240B and is centered on the axis B. The top of the post 240C is in the form of a circular flange 240D having a planar top surface. The outside diameter of the flange is greater than the inside diameter of the passageway 224M extending through the line suction port 224A. The post 240D incudes a central bore configured to slidably receive the post 238D of the float. With the float mounted within the basket as shown in FIG. 12, if the level of urine reaches the bottom of the basket, the urine will enter the openings 238C of the basket and cause the float to move upward in the basket guided by the engagement of the posts 238A and 240C. If the level of urine in the canister reaches the height to cause the float to rise to the height wherein its flange 240D engages the surface 224N of the lid contiguous with the bottom of the passageway 224M, that action will seal or occlude the passageway to thereby prevent any urine from entering it.

It should be noted at this juncture that other means can be used in lieu of the isolation valve 228 to prevent any urine in the canister 222 from entering into the passageway 224M of the line suction port 224A. A few examples of such other means that are contemplated by this invention are: a hydrophobic filter, a semipermeable foam member to allow air but not urine to pass therethrough, or an expandable impermeable foam member which expands when exposed to urine to completely occlude the line suction port. The expandable impermeable foam member may be of conventional construction like that used in commercially available canisters coupled to a hospital's line suction port or line suction regulator to prevent materials collected in the receptacle from gaining egress out of the canister and into the hospital's suction system or line suction controller. For example, the filter may comprise a short section of closed-cell foam or other material which expands in the presence of a liquid. The foam section is mounted within a collar projecting downward from the lid into the hollow interior of the canister so that a space or passageway is located between the collar and the foam section. The passageway is normally in fluid communication between the interior of the canister and the outlet port 224A. The foam member is operative so that if and when the level of urine collected in the canister reaches the foam member it will expand, thereby closing the passageway between it and the collar so that the outlet port will be closed, thereby precluding any urine from gaining egress from the canister into the suction line.

As mentioned earlier the lid 224 of the unit 220 includes a spout to enable urine collected within the canister 222 to be poured out to empty the canister. That spout is designated by the reference number 2240 and is in the form of a circular wall projecting upward from the top surface 224D of the lid and diametrically opposed to the regulated suction port 226B. The spout includes a central passageway 224P extending through it from the top of the spout to the bottom of the spout. The passageway is in fluid communication with the hollow interior 222A of the canister 222. The spout is normally closed or sealed by the cap 230, like shown in FIG. 12.

In accordance with one exemplary preferred embodiment of the suction regulator 226, inner diameter of the lower chamber 224G is approximately 1.5 inches. The inner diameter of the upper chamber 224L is approximately 1.5 inches. The spring 236 is configured to naturally apply a bias force of approximately 2.5 pounds. The inner diameter of the internal port 226A is approximately 0.5 inches. The inner diameter of the passageway 224F is approximately 0.25 inches. The opening located within the bounds of the valve seat 224I is approximately 0.2 inches. The atmospheric reference port 232C is approximately 0.044 inches in diameter. Each tubing section 24 and 26 is conventional having an internal passageway of approximately 0.25 inches in diameter, and each section is approximately six feet in length, but could be shorter or longer depending upon the application.

With a suction regulator 226 sized as just described, in a system like that described during typical operation the flow rate of air can be up to 100 SCFH as compared to the 15 SCFH rate observed with some commercially available wall regulator set to the suggested 40 mmHg. The additional flow allows for increased urine capture at the interface of the actual catheter, faster drying of the catheter (which helps prevent skin breakdown and infection) and pulls the urine through the tubing into the canister 222 more efficiently. This is especially true if the tubing drapes down below the height of the patient and canister.

As should be appreciated by those skilled in the art since the suction regulator 226 has a fixed regulated set-point value, users of the system 200 do not have to pick and set a particular value for the suction to be applied to the patient's external catheter 22. As such a safe level of suction will be automatically be applied to the patient by the suction regulator 226 of the unit 220 without requiring hospital personnel or other operators to set the desired value of suction to be applied by the external catheter. Moreover, since the suction regulator 226 has a fixed regulated set-point value, there is no need for the integrated urine collection canister and suction regulator unit 220 to include any dials or other indicators to provide the hospital personnel or other operators with a reading of the suction level being applied so that they could set the regulator to the desired set-point value. Accordingly, integrated urine collection canister and suction regulator units constructed in accordance with this can be simple in construction, low in cost and easy to use.

It must be pointed out at this juncture that the various components of the systems shown and described above are merely exemplary of various components that may be used in accordance with this invention to provide the capabilities as discussed above. For example, the suction regulator 26 may be constructed so that it includes a piston coupled to the central portion of the diaphragm, with the piston including a sealing disk configured to engage the valve seat, instead of the sealing disk portion of the diaphragm engaging the valve seat. Moreover, the suction regulator 26 need not be mounted onto the receptacle or canister. In that case, the suction tube 42 may be omitted and a section of flexible tubing connected between the port 30B of the receptacle or canister and the tubular extension 36H. Further still, some hospitals in which the subject system will be used have special regulator set-ups that allow for connection of a suction canister directly below the wall regulator. In such a case the tubing section 28 of the system 100 of this invention may be omitted. Also, it should be pointed out that the systems of this invention are not limited to use in hospitals, but can be used in any application providing care to a female patient be she human or animal. In fact, the subject invention is not limited for use with females. In this regard, it is contemplated that the subject invention can be used with external catheters for male patients, be they human or animal.

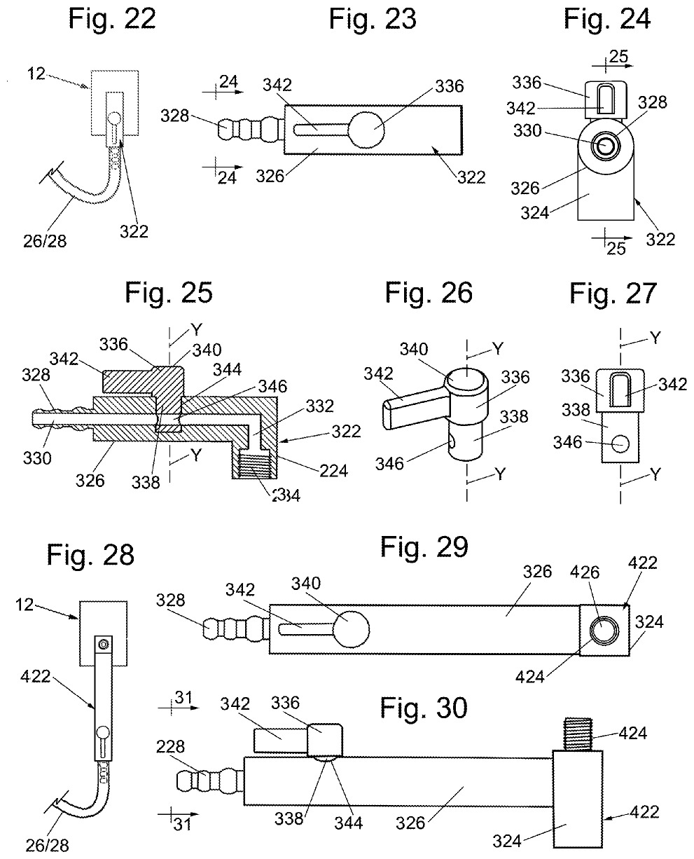

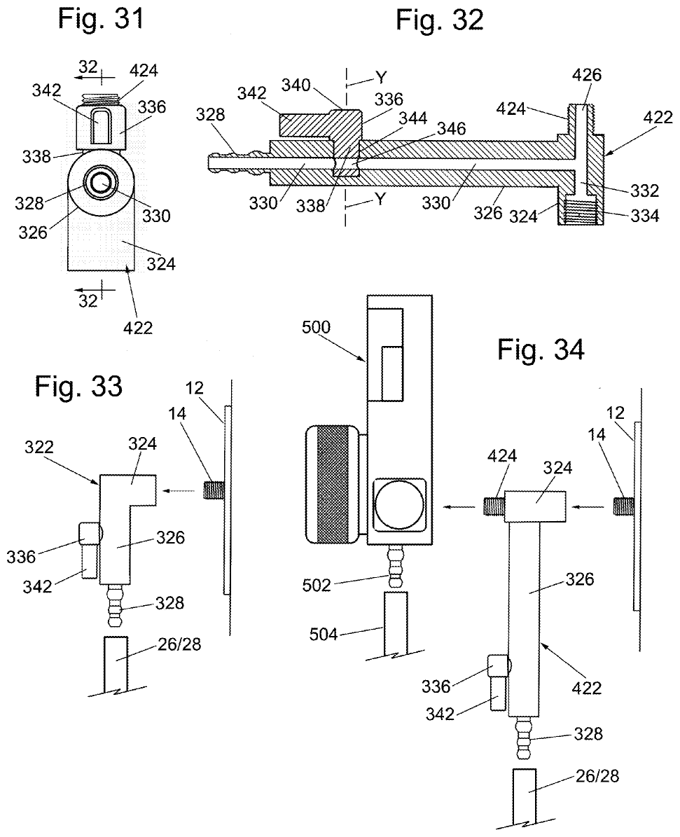

The systems 100 and 200 as described above may make use of an adapter and an adapter/splitter, like those disclosed and claimed in our aforementioned U.S. patent application Ser. No. 16/833,980, whose disclosure is incorporated by reference herein, for connecting those systems to the hospital's line suction port connector. One such adapter 322 is shown in FIGS. 22-27 and 33. One such adapter/splitter is the adapter/splitter 422 shown in FIGS. 28-32 and 34.

The adapter 322 is configured to mount connect the system 100 or 200 to the line suction port 12 of a hospital or some other facility providing suction to a patient, like shown in FIG. 33. As can be seen in that figure, the line suction port 12 includes a conventional externally threaded connector 14. The connector 14 has a passageway (not shown) through which suction from the hospital's line suction source is provided.

The adapter 322 includes a generally L-shaped body formed of any suitable material, e.g., plated brass. The body of the adapter includes a base section 324 and an elongated section 326. The distal end of the elongated section 326 is in the form of a conventional bubble barb 328 for receipt of the proximal end of the tubing section 26 or 28. The elongated section 326 is of a generally circular profile when viewed from the distal end thereof. A generally L-shaped passageway is located in the body of the adapter 322. The L-shaped passageway includes a linear passageway section 330 extending through the bubble barb and through the elongated section centered on the central longitudinal axis of the elongated section. The proximal end of the passageway section 330 merges with and is in fluid communication with a passageway section 332. The passageway section 332 extends perpendicularly to the passageway section 330 and terminates in a larger diameter internally threaded bore 334. The internally threaded bore 334 serves as the inlet to the adapter 322 and is configured to receive the external threads of the hospital's wall connector 14 to mount the adapter 322 onto the line suction port 12. The internal diameter of the passageway section 332 preferably matches the internal diameter of the passageway extending through the line suction connector 14, e.g., 0.2 in. The internal diameter of the passageway section 330 is the same as the internal diameter of the passageway section 332.

The adapter 322 includes a valve 336 configured to be in either an open or closed position. When in the closed position (to be described shortly) the valve closes or blocks the passageway section 330 to isolate the passageway in the bubble barb 328 from the line suction provided at the connector 14, When the valve is open the passageway section 330 is unblocked so that the line suction provided at the connector 14 appears at the passageway in the bubble barb and hence is provided to the external catheter urine collection system 100 or 200. The valve 336 is best seen in FIGS. 26 and 27 and is a rotatable integral member, formed of any suitable material, e.g., plated brass. The valve basically comprises a cylindrical shaft 338, a hub 340 and a handle 342. The cylindrical shaft 338 is located within a correspondingly shaped cylindrical bore 344 (FIG. 25) intersecting the passageway section 330 perpendicularly thereto. A central axis Y extends through the bore 344 and serves as the rotation axis about which the shaft 338 of the valve rotates when the valve is moved from its closed state to its open state, and vice versa. The shaft 338 includes a hole 346 extending diametrically through it. The internal diameter of the hole 346 is the same as the internal diameter of the passageway section 330. The upper end of the shaft terminates in the hub 340, which is of slightly larger diameter than the diameter of the shaft. A generally planar handle 342 projects radially outward from the hub. The handle is configured to be rotated either clockwise or counterclockwise about the axis Y to either open the valve or close it. In particular, when the handle is rotated to the position wherein the hole 346 of the valve is axially aligned with the passageway 330 (e.g., the handle extending parallel to the elongated section 326), the valve will be in its open state so that suction is applied from the passageway section 332 to the passageway section 330 in the bubble barb 328. When the handle of the valve is rotated to the position wherein no portion of the hole 346 of the shaft is in communication with the passageway 330 (e.g., the handle extending perpendicular to the elongated section 326) suction appearing at the connector 14 will be isolated from the system 100 or 200.

Inasmuch as the adapter 322 when mounted on the connector 14 of the port 12 to connect the external catheter urine collection system 100 or 200 to the line suction at the port will take that port out of service for uses other than removing urine from the patient via the external catheter 22, the adapter/splitter 422 like shown in FIG. 28 is provided to enable the port 12 to be used with another suction device at the same time it is used with the system 100 or 200. In particular, as best seen in FIG. 34, the adapter/splitter 422 is configured to connect the external catheter urine collection system 100 or 200 the threaded connector 14 of line suction port 12 of a hospital (or some other facility providing suction to a patient). The adapter/splitter 422 is in many ways identical in construction to the adapter 322. Thus, the common features of adapter/splitter 422 with the adapter 322 will be given the same reference numbers and the details of their construction and operation will not be reiterated in the interest of brevity. The adapter/splitter also includes an externally threaded connector 424 having a passageway 330 extending through the elongated section 326. The externally threaded connector 426 and its elongated section 326 together form the splitter portion of the adapter/splitter 422, thus providing another site at which the hospital's line suction is available. For example, as will be described shortly the externally threaded connector 424 can serve as the means for connecting and mounting a conventional suction regulator 500 thereon to control suction to some other device or equipment serving the patient.

The top of the base section 324 of the adapter/splitter 422 includes the heretofore identified externally threaded connector 424. As best seen in FIG. 32, a passageway section 426 is centered in the base section 324 and is coaxial and aligned with the passageway section 332 so that the passageway sections 426 and 332 are in fluid communication with each other. As such passageway section 426 serves to bring suction from the line suction connector 14 to any device that may be connected to the externally threaded connector 424 irrespective of whether or not the valve 336 of the adapter/splitter 422 is open or closed. The internal diameter of the passageway section 426 is preferably the same as the internal diameter of the passageway sections 332 and 330.

Turning now to FIG. 34 the connector 424 of the adapter/splitter 422 is shown in the process of being connected to an internally threaded bore (not shown) of an exemplary suction regulator 500. The exemplary suction regulator 500 shown in FIG. 34 is a conventional suction regulator like that sold by Boehringer Laboratories, LLC, the assignee of the subject invention, under the model designation 3844. Other suction regulators available from the assignee of this invention as well as other manufacturers can be used by being connected to the threaded connector 424. Thus, while the system 100 or 200 is connected to the bubble barb 328 so that it can be used to withdraw urine from a patient, suction can be provided from the suction regulator 500 mounted on the connector 424 of the adapter/splitter 422 to provide suction for some other use. To that end, the suction regulator 500 includes a bubble barb 502 for disposition within an open proximal end of a tubing section 504, which will provide suction for some other application for the patient.

Various other changes can be made to systems of this invention, in addition to changes to the integrated urine collection canister and suction regulator unit or any of its components. For example, some hospitals in which the subject system will be used have special regulator set-ups that allow for connection of a suction canister directly below the wall regulator. In such a case the tubing section 26 of the system 200 of this invention may be omitted so that the line suction port 224A of the integrated urine collection canister and suction regulator unit is directly connected to the wall regulator. Also, it should be pointed out that the systems and the integrated suction regulator and urine collector unit of this invention are not limited to use in hospitals, but can be used in any application providing care to a female patient, be she human or animal. In fact, the subject invention is not limited for use with females. In this regard, it is contemplated that the subject invention can be used with external catheters for male patients, be they human or animal.

Without further elaboration the foregoing will so fully illustrate our invention that to others may, by applying current or future knowledge, adopt the same for use under various conditions of service.

We claim:

1. A system for automatically removing by suction urine voided by a patient comprising:
an external catheter configured for external disposition in fluid communication with a urethra opening of the patient, whereupon urine voided by the patient is received by said external catheter;
a receptacle or canister for collecting urine and configured to be coupled to a source of suction providing suction having a first value;
a suction regulator having a regulated set-point value to which said suction regulator is fixedly set, said suction regulator being coupled to said receptacle or canister and the source of suction to regulate the amount of suction from said first value to said regulated set-point value, said regulated set-point value being lower than said first value, whereupon operation of said suction regulator applies regulated suction at said fixed regulated value to said external catheter to result in urine from said external catheter being carried through said suction regulator and into said receptacle or canister, said suction regulator comprising a regulated suction port configured to be coupled to said external catheter to apply regulated suction at said fixed regulated value to said external catheter, a line suction port configured to be coupled to said receptacle or canister and to the source of suction, a movable diaphragm, a valve seat configured to be engaged by a movable sealing member coupled to said movable diaphragm, and a passageway located at said valve seat and providing a straight fluid path to said line suction port.

2. The system of claim 1, wherein said receptacle or canister comprises a first port, a second port, and a hollow interior in fluid communication with said first and second ports, said first port being configured to be connected to the source of suction, and wherein said line suction port of said suction regulator is configured for coupling to said second port of said receptacle or canister, said suction regulator being configured to carry urine from said external catheter by said regulated suction through said suction regulator and into said second port of said receptacle or canister for collection in said hollow interior.

3. The system of claim 2, wherein said suction regulator additionally comprises a first chamber, a second chamber, and a biasing member, said first chamber being configured to have suction applied thereto from said canister, said second chamber being at atmospheric pressure, said movable diaphragm separating said first chamber from said second chamber, whereupon a differential pressure exists between said first and second chambers, said differential pressure imparting a differential pressure force on said movable diaphragm, said biasing member being configured to impart a counter force on said movable diaphragm that opposes said differential pressure force, said movable sealing member comprising a sealing disk coupled to said movable diaphragm.

4. The system of claim 3, wherein said valve seat and said movable sealing member coupled are located in said first chamber, said valve seat surrounding an opening for fluids within said first chamber to flow therethrough into said passageway, said sealing member blocking said opening when said differential pressure force exceeds said counter force imparted by said biasing member.

5. The system of claim 4, wherein said suction regulator additionally comprises a housing in which said first and second chambers are located, and a hollow hub located in said first chamber, said valve seat and said passageway being located in said hub.

6. The system of claim 1, wherein said urine is carried through said suction regulator and into said receptacle or canister by air which is flowing at a high flow rate up to approximately 100 standard cubic feet per hour (SCFH)

7. The system of claim 1, wherein said regulated value of suction is within the range of approximately 40-80 mmHg.

8. The system of claim 5, wherein said second chamber is maintained at said atmospheric pressure by a bleed hole in said housing.

9. The system of claim 8, wherein said bleed hole is located within a recess in said housing, said recess having at least one opening at an end thereof to prevent blockage of said bleed hole.

10. The system of claim 1, wherein said suction regulator is mounted on said receptacle or canister.

11. The system of claim 1, additionally comprising an adapter configured to be coupled between the source of suction and said receptacle or canister, said adapter comprising an inlet, a valve and a first connector, said inlet being configured to be connected to the source of suction, said first connector being configured to be connected to said receptacle or canister, said valve being configured to be in either a closed state or an open state, whereupon when in said closed state said receptacle or canister is isolated from said inlet, and when in said open state suction at said first value is provided from said inlet to said first connector and to said receptacle or canister.

12. The system of claim 11, wherein said adapter includes an internal passageway having one end in communication with said inlet and another end in communication with said first connector, and wherein said valve includes a rotatable portion having an opening extending therethrough, said rotatable portion intersecting said internal passageway, whereupon when said rotatable portion is in a first rotatable position said rotatable portion blocks said internal passageway between said inlet and said first connector, and when said rotatable portion is in a second position said opening unblocks said internal passageway between said inlet and said first connector to enable suction at said first value to appear at said first connector.

13. The system of claim 11, wherein said first connector is configured to have a first section of suction tubing having a first end connected to it and a second end connected to said receptacle or canister.

14. The system of claim 11, wherein said adapter additionally comprises a splitter including a second connector having a passageway section in fluid communication with said internal passageway between said inlet and said valve, whereupon suction at said first value is available from said inlet to said second connector irrespective of the state of said valve.

15. A system for automatically removing by suction urine voided by a patient comprising:
an external catheter configured for external disposition in fluid communication with a urethra opening of the patient; and
an integrated suction regulator and urine collector unit coupled to said external catheter, said integrated suction regulator and urine collector unit comprising:
a receptacle or canister configured for collecting urine therein;
a suction regulator mounted on said receptacle or canister and being configured for applying controlled suction to said external catheter from a source of suction having a first value, said suction regulator having a regulated set-point value to which the suction regulator is fixedly set, said suction regulator being configured to regulate the amount of suction from said first value to said regulated set-point value, said regulated set-point value being lower than the first value, whereupon operation of said suction regulator applies regulated suction at said fixed regulated value to said external catheter to result in urine from the external catheter being carried through said suction regulator and into said receptacle or canister.

16. The system of claim 15, wherein said urine is carried through said suction regulator and into said receptacle or canister by air which is flowing at a high flow rate up to approximately 100 SCFH.

17. The system of claim 15, wherein said receptacle or canister comprises a first port and a hollow interior in fluid communication with said first port, said first port of said receptacle or canister being configured to be connected to the source of suction, said suction regulator comprising a first port and a second port, said second port of said suction regulator being connected to and in fluid communication with said hollow interior of said receptacle or canister and the source of suction, said suction regulator being configured for providing said regulated suction at said first port of said suction regulator, said first port of the suction regulator being configured to be coupled to said external catheter to carry urine from said external catheter by said regulated suction through said suction regulator and into said hollow interior of said receptacle or canister for collection therein.

18. The system of claim 15, wherein said receptacle or canister comprises a hollow body and a lid disposed over said hollow body, said suction regulator being mounted on said lid.

19. The system of claim 15, wherein said regulated value of suction is within the range of approximately 40-80 mmHg.

20. The system of claim 15, wherein said suction regulator comprises a first chamber, a second chamber, a movable diaphragm and a biasing member, said first chamber being configured to have suction applied thereto from said receptacle or canister, said second chamber being at atmospheric pressure, said movable diaphragm separating said first chamber from said second chamber whereupon a differential pressure exists between said first and second chambers, said differential pressure imparting a differential pressure force on said movable diaphragm, said biasing member being configured to impart a counter force on said movable diaphragm that opposes said differential pressure force.

21. The system of claim 20, wherein said first chamber comprises a valve seat and a movable sealing member coupled to said movable diaphragm, said valve seat surrounding an opening for fluids within said first chamber to flow therethrough, said sealing member blocking said opening when said differential pressure force exceeds said counter force imparted by said biasing member.

22. The system of claim 21, wherein said second chamber is maintained at said atmospheric pressure by a bleed hole in said housing located within a recess having at least one opening at an end thereof to prevent blockage of said bleed hole.

23. The system of claim 15, additionally comprising an adapter configured to be coupled between the source of suction and said receptacle or canister, said adapter comprising an inlet, a valve and a first connector, said inlet being configured to be connected to the source of suction, said first connector being configured to be connected to said receptacle or canister, said valve being configured to be in either a closed state or an open state, whereupon when in said closed state said receptacle or canister is isolated from said inlet, and when in said open state suction at said first value is provided from said inlet to said first connector and to receptacle or canister.

24. The system of claim 23, wherein said adapter includes an internal passageway having one end in communication with said inlet and another end in communication with said first connector, and wherein said valve includes a rotatable portion having an opening extending therethrough, said rotatable portion intersecting said internal passageway, whereupon when said rotatable portion is in a first rotatable position said rotatable portion blocks said internal passageway between said inlet and said first connector, and when said rotatable portion is in a second position said opening unblocks said internal passageway between said inlet and said first connector to enable suction at said first value to appear at said first connector.

25. The system of claim 23, wherein said first connector is configured to have a first section of suction tubing having a first end connected to it and a second end connected to said receptacle or canister.

26. The system of claim 23, wherein said adapter additionally comprises a splitter including a second connector having a passageway section in fluid communication with said internal passageway between said inlet and said valve, whereupon suction at said first value is available from said inlet to said second connector irrespective of the state of said valve.

27. A method for automatically removing by suction urine voided by a patient comprising:
providing an external catheter in fluid communication with a urethra opening of the patient, whereupon urine voided by the patient is received by said external catheter;
providing a source of suction having a first value;
providing an integrated suction regulator and urine collector unit comprising a suction regulator and a receptacle or canister for collecting urine, said suction regulator having a regulated set-point value to which said suction regulator is fixedly set and which is lower than said first value;
coupling said integrated suction regulator and urine collector unit to said source of suction; and
operating said suction regulator to automatically regulate the amount of suction from said first value to said regulated set-point value and applying suction at said regulated set-point value to said external catheter, whereupon urine from said external catheter is carried through said suction regulator and into said receptacle or canister.

28. The method of claim 27, wherein said urine is carried through said suction regulator by air which is flowing at a high flow rate up to approximately 100 (SCFH).

29. The method of claim 27, wherein said regulated value of suction is within the range of approximately 40-80 mmHg.
